(19) United States
(12) Patent Application Publication
(10) Pub. No.: US 2011/0208319 A1
(43) Pub. Date: Aug. 25, 2011

(54) BLOOD FILTERING DEVICE AND METHOD

(75) Inventor: Morris LASTER, Jerusalem (IL)

(73) Assignee: Clil Medical Ltd., Jerusalem (IL)

(21) Appl. No.: 13/101,189

(22) Filed: May 5, 2011

Related U.S. Application Data
(63) Continuation of application No. PCT/IL2009/001031, filed on Nov. 4, 2009.
(60) Provisional application No. 61/111,744, filed on Nov. 6, 2008.
(60) Provisional application No. 61/111,743, filed on Nov. 6, 2008.
(60) Provisional application No. 61/111,742, filed on Nov. 6, 2008.

(51) Int. Cl. A61F 2/04

(52) U.S. Cl. 623/23.65

(57) ABSTRACT

A device for filtering blood is provided herein. The device includes a device body configured for at least partial implantation within a marrow of a bone and a filter disposed on or within the device body and being for filtering blood flowing through the marrow.

### RELATED APPLICATIONS

This application is a continuation of PCT Patent Application No. PCT/IL2009/001031 having International filing date of Nov. 4, 2009, which claims the benefit of priority under 35 USC 119(e) of U.S. Provisional Patent Application Nos. 61/111,744, 61/111,743 and 61/111,742 all filed Nov. 6, 2008. The contents of all of the above applications are incorporated by reference as if fully set forth herein.

### FIELD AND BACKGROUND OF THE INVENTION

The present invention relates to a device for filtering biological fluids and more specifically to a self cleaning filtering device which is capable of removing excess water from circulating plasma.

The kidneys filter the blood and remove excess fluid, minerals, and wastes. They also produce hormones that maintain bone strength and blood homeostasis. When kidneys fail to perform these normal functions, harmful wastes build up in the body, the blood pressure may rise, and the body may retain excess fluid and not produce sufficient amounts of red blood cells. As such, kidney failure requires treatment in order to correct or compensate for such failure.

End-Stage Renal Disease (ESRD) occurs when the kidneys are no longer able to function at a level that is necessary for day-to-day life, up to the point where kidney function is less than about 10% of a normal, disease-free kidney. An abnormally low glomerular filtration rate is usually determined indirectly by the creatinine level in the blood serum. The most common cause of ESRD is diabetes. Symptoms of ESRD can include, for example, unintentional weight loss, nausea or vomiting, general ill feeling, fatigue, headache, decreased urine output, easy bruising or bleeding, blood in the vomit or stools, elevated blood urea nitrogen (BUN) levels and decreased creatinine clearance.

Since ESRD patients are not capable of sufficiently excreting fluids, water and other fluids remain in the body until they are removed by ultrafiltration (removal of excess fluid from the body) during dialysis. Consequently, as the kidney function decreases the fluid volume overloads and the blood pressure is increased.

Another major cause of fluid overload that may benefit from ultrafiltration is congestive heart failure (CHF) secondary to ischemic heart disease, atherosclerotic or genetic disorders. Due to cardiac pump failure, the kidneys have diminished flow which results in increased blood pressure and fluid overload.

Dialysis is performed routinely on individuals suffering from acute or chronic renal failure, have ESRD or CHF related fluid overload. The process involves removing waste substances and fluid from the blood that are normally eliminated by the kidneys. Dialysis may also be used in individuals exposed to toxic substances in order to prevent renal failure from occurring.

There are two types of dialysis that may be performed: hemodialysis and peritoneal dialysis.

Hemodialysis involves fluid removal through ultrafiltration, causing free water and some dissolved solutes to move across the membrane along a created pressure gradient. Hemodialysis utilizes counter current flow, which maintains the concentration gradient across the membrane at a maximum and increases the efficiency of the dialysis. The blood is taken by a special type of access, called an arteriovenous (AV) fistula, which is placed surgically, usually in the arm. After access has been established, the blood drains though a large hemodialysis machine which bathes the hemofiltration cartridge in a special dialysate solution that adjusts solute concentration and removes waste substances and fluid. The “clean” blood is then returned to the bloodstream. Hemodialysis is usually performed three times a week with each treatment lasting from 3 to 5 or more hours. Because proper maintenance of hemodialysis equipment (e.g. membranes, pumps) is critical, hemodialysis sessions are often performed at a treatment center. Possible complications of hemodialysis can include muscle cramps and low blood pressure caused by removing too much fluid and/or removing fluid too rapidly.

Peritoneal dialysis uses the peritoneal membrane to filter the blood. Peritoneal dialysis is performed by surgically placing a special, soft, hollow tube into the lower abdomen near the navel. A mixture of minerals and sugar dissolved in water, called dialysate solution, is instilled into the peritoneal cavity and is left in the abdomen for a designated period of time in which the dialysate fluid absorbs the waste products, toxins and extra water through the peritoneum membranes. After several hours, the used solution containing the wastes from the blood is drained from the abdomen through the tube. Then the abdomen is refilled with fresh dialysis solution, and the cycle is repeated. The process of draining and refilling is called an exchange. Patients usually undergo four to six exchanges of the dialysis solution per day. An infection of the peritoneum, or peritonitis, is the most common problem of peritoneal dialysis.

Although dialysis is a common procedure it suffers from several disadvantages, including fluids balance impairment, the need of special diet, high blood pressure, psychological problems because of the change in the life style due the need to go to the dialysis treatment several times a week for several hours each time.

Several attempts have been made to devise systems which overcome at least some of the aforementioned limitations of dialysis devices. U.S. Pat. Nos. 5,037,385 and 10/922,478 disclose implantable peritoneal dialysis devices. The aforementioned system described includes an implantable peritoneourinary pump system and an implantable dialysate infusion system. When in use, the device has a semi-permeable reservoir implanted in the peritoneal cavity. The reservoir receives blood waste and drains through one or more conduits via a pump to the biological bladder, which is a complicated arrangement.

U.S. Pat. No. 5,902,336 describes another implantable system which employs an ultrafiltration device for removing low to medium molecular weight solutes and fluids from the blood of a patient experiencing renal failure. In this system the fluid flows between the patient's vascular system, through an access to the artery and/or vein, and the patient's bladder or urethra. As such, this systems requires surgical attachment of a metal or hard plastic device, to a soft biological tissue (artery or vein), a procedure which often results in undesirable side effects such as vessel shearing/tearing, clotting, fibrosis, infection and thrombosis. Moreover such a system requires installing a pump inside the patient's body.

Dialyzing kidney-like functions based on transplanted or implanted biologically active cells or tissues have also been considered, and bone marrow has been postulated as a potentially suitable site due to it's ability to tolerate foreign antigens and it's access to the circulatory system.

The bone marrow is an immunoprivileged site and thus can be utilized for the introduction of materials foreign to the host. Such an example is disclosed in U.S. Pat. No. 6,463,933, describing a method for delivering a biologically active substance including: cells, tissues, nucleic acids, vectors, proteins or pharmaceutical compositions to a mammal, by introducing the substance into the bone or bone marrow. An embodiment alluded to in '933 refers to the implantation of kidney cells into the bone marrow for dialysis, but such a system is far from being achieved.

Non biological implants which do not have the drawbacks outlined above such as clotting, infection, tissue necrosis, tumor formation and thrombosis, and avoid tearing problems remain a long felt and unmet need.

While reducing the present invention to practice, the present inventors have devised a non-biological dialysis device which is designed for implantation in the bone marrow of a patient and provide ultrafiltration of blood plasma thereby overcoming the abovementioned limitations of prior art devices.

### SUMMARY OF THE INVENTION

According to one aspect of the present invention there is provided a filtration device (BFD) which is implantable within the bone marrow of a patient. The device includes a device body (formed as a collector or cartridge) which is configured for implantation within the bone marrow of a subject and a filter material disposed within or on the device body. The device body is configured for directing bone marrow blood through the filter such that waste substances are retained by the filter and water and solutes are passed therethrough, collected by the device body and removed out of the blood.

According to further features in preferred embodiments of the invention described below, the device further includes a conduit for directing water and solutes removed from the blood to the bladder, Genito-Urinary (GU) system or a reservoir.

According to still further features in the described preferred embodiments the device maintains a pressure gradient between the blood flow in the bone marrow and the filter material. The pressure gradient is sufficient to direct excess water to the bladder or the GU system whilst retaining defined macromolecules.

According to still further features in the described preferred embodiments the filter material is selected from compositions suitable for extracting water from circulating bone marrow blood flow.

According to still further features in the described preferred embodiments the device additionally comprises a cage accommodating the collector/cartridge.

According to still further features in the described preferred embodiments the cage adapted to protect the collector/cartridge from ingrowths of bone tissue.

According to still further features in the described preferred embodiments the cage comprising titanium and/or hydroxyapatite adapted for stimulating cancellous growth into the collector/cartridge, thereby promoting osteointegration of the collector/cartridge.

According to still further features in the described preferred embodiments the cage coated by titanium and/or hydroxyapatite adapted for stimulating cancellous growth thereby promoting osteointegration of the collector/cartridge.

According to still further features in the described preferred embodiments the conduit/tube in fluid communication with the implantable filter material; the conduit/tube comprising fluid communication means to transport collected water to the patient's bladder or GU system.

According to still further features in the described preferred embodiments the device is adapted for implantation in a bone marrow site within a bone, the bone selected from a group consisting of long bones, preferably bones adjacent to or positioned above the patient bladder or GU system, including the iliac crest, rib, sternum, hip, bones of the lower arm, and bones of the upper arm.

According to still further features in the described preferred embodiments the conduit/tube adapted to allow gravity assisted flow to the bladder/GU system.

According to still further features in the described preferred embodiments the filter material is capable of withstanding a fluid pressure of about 200 mm Hg.

According to still further features in the described preferred embodiments the device is adapted by size, shape, materials, and type of filter composition for implantation in a location within the bone marrow such that sufficient gradient pressure for filtering fluids without assistance from a pump is achieved.

According to still further features in the described preferred embodiments the filter material has a minimum surface area of 0.5 cm2.

According to still further features in the described preferred embodiments the filter material has a molecular cut off between about 5 KDa and about 50 KDa.

According to still further features in the described preferred embodiments the filter material is at least partially comprised of metal.

According to still further features in the described preferred embodiments the metal is electrically and/or magnetically charged.

According to still further features in the described preferred embodiments the metal is negatively charged in order to repel protein fouling.

According to still further features in the described preferred embodiments the metal which is paramagnetic.

According to still further features in the described preferred embodiments the metal comprises biocompatible materials selected from a group consisting of single or multiple stainless steel alloys, nickel titanium alloys, cobalt-chrome alloys, molybdenum alloys, tungsten-rhenium alloys, or any combination thereof.

According to still further features in the described preferred embodiments the filter material includes a biocompatible polymeric substance.

According to still further features in the described preferred embodiments the filter is pleated, folded, cylindrical, conical, spiral, scrolled or a planar sheet or hollow fibers, or any combination thereof.

According to still further features in the described preferred embodiments the biocompatible polymeric substance which is a physiologically acceptable substance selected from a group consisting of polyacrylonitrile, polysulfone, polyethersulfone, poluethylene, polymethylmethacrylate, polytetrafluoroethylene, polyester, polypropylene, polyether ether ketone, Nylon, polyether-block co-polyamide polymers, polyurethanes such as aliphatic polyether polyurethanes, polyvinyl chloride, thermoplastic, fluorinated ethylene propylene, cellulose, collagen, silicone or any combination thereof.

According to still further features in the described preferred embodiments the conduit/tube having a diameter ranging between about 1 to about 30 mm and about 5 to about 10 mm.

According to still further features in the described preferred embodiments the conduit/tube having length ranging between about 5 to about 40 cm and about 10 to about 20 cm.

According to still further features in the described preferred embodiments the device body is made of biocompatible materials selected from a group consisting of polyester, polypropylene, PTFE, ePTFE, PEEK, Nylon, polyether-block co-polyamide polymers, polyurethenes such as aliphatic polyether polyurethanes, PVC, PAN, PS, polyethersulfone, polyethylene, polymethylmethacrylate (PMMA), polyhydroxylmethylmethacrylate (PHMMA), thermoplastic, FEP, cellulose, extruded collagen, silicone or any combination thereof.

According to still further features in the described preferred embodiments the conduit/tube connects to the bladder and/or GU system to facilitate excretion of fluids.

According to still further features in the described preferred embodiments the device includes a self clearing mechanism for clearing the filter.

According to still further features in the described preferred embodiments the device further comprises a pump system, providing high pressure gradients and flow rates.

According to still further features in the described preferred embodiments the pump also self-clears the filter from fouling substances.

According to still further features in the described preferred embodiments the filter is provided with a mechanism for continuous unclogging of protein and lipid clots.

According to still further features in the described preferred embodiments the mechanism is a bolus mechanism adapted to provide a peristaltic/pumping function for dislodging the clots; a micro-propeller mechanism adapted to provide a positive and negative pressure for increasing hydrostatic pressure improving filtration and dislodging the clots; a scrubber mechanism adapted to peel filter clogging from a side surface of the filter; reinforcing mechanism comprising energizable relaxer piezoelectric threads; the threads are adapted for electrically-induced vibrating for periodically clearing the filter; a mechanism comprising at least two cylinders or sheets adapted for moving against each other such that the filter pores are cleared; a sponge like mechanism for actively clearing the filter; electrodialysis reversal mechanism for reversing an electrical current across the assembly thereby removing the fouling and scaling constituents from one cycle to the next.

According to still further features in the described preferred embodiments the device further comprises a chargeable power source.

According to still further features in the described preferred embodiments the filter is cylindrically configured and the scrubber is configured in a shape of a ring adapted for peeling the filter clogging from the side surface of the cylindrically configured filter.

According to still further features in the described preferred embodiments the scrubber includes a wire begirding the side surface of the filter structure; the wire is made of a shape memory alloy; the wire is adapted for reversibly extending along the side surface for peeling the filter clogging.

According to still further features in the described preferred embodiments the filter adapted for reversibly changing a form thereof.

According to still further features in the described preferred embodiments the device includes a sponge like mechanism for actively inducing flow and filter clearing by twisting, wringing or squeezing.

According to still further features in the described preferred embodiments the device further comprises an electrodialysis reversal mechanism including a voltage transducer device for reversibly changing the electrical potential difference across the membrane thereby facilitating unclogging of the filter.

According to still further features in the described preferred embodiments the pump system includes: (a) a micro-pump for creating a positive pressure in the permeate lumen; (b) a micro-valve for reversibly sealing off the permeate lumen; (c) a controller for controlling the micro-pump and micro-valve according to a predetermined protocol; and, (d) an electric battery for energizing the micro-pump, micro-valve, and controller.

According to still further features in the described preferred embodiments the micro-pump is adapted for inducing negative pressure impulses in the permeate lumen of the device body.

According to still further features in the described preferred embodiments the filter includes at least one water permeable membrane sheet. The membrane sheet has a first side and a second side, such that (i) a retentate lumen interleaves the first side layers of the membrane sheet; and, (ii) a permeate lumen interleaves the second side layers of the membrane sheet.

According to still further features in the described preferred embodiments the device further comprises a biocompatible hydrophilic material partially dispersed or otherwise immobilized within the permeate lumen side of the membrane sheet; the material adapted to provide an increased hydrostatic pressure gradient across the membrane greater than the gradient obtained across same membrane in the absence of the material.

According to still further features in the described preferred embodiments the device body includes at least one retentate lumen exposed to circulating blood.

According to still further features in the described preferred embodiments the device body includes at least one leak proof manifold which is in an effluent connection with the permeate lumen.

According to still further features in the described preferred embodiments the filter is disposed within a filter cartridge including (a) a plurality of rods having elongated hollow membrane fibers longitudinally arranged; (b) at least one permeate lumen in liquid contact to outer walls of the fibers; (c) a plurality of retentate lumens disposed within the hollow fibers; (d) the biocompatible hydrophilic material dispersed within the at least one permeate lumen.

According to still further features in the described preferred embodiments the cartridge assembly comprises a solid rod membrane arrangement, further wherein exterior walls of the rods form the retentate lumen and interior of the rods form the permeate lumen.

According to still further features in the described preferred embodiments the interior of the solid membrane rods at least partially comprises hydrophilic material providing increased hydrostatic pressure gradient between the permeate lumen and the retentate lumen.

According to still further features in the described preferred embodiments the membrane comprises biocompatible polymeric substance selected from a group consisting of polyacrylonitrile, polysulfone, polyethersulfone, poluethylene, polymethylmethacrylate, polytetrafluoroethylene, polyester, polypropylene, polyether ether ketone, Nylon, polyether-block co-polyamide polymers, polyurethanes such as aliphatic polyether polyurethanes, polyvinyl chloride, thermoplastic, fluorinated ethylene propylene, cellulose, collagen, silicone or any combination thereof.

According to still further features in the described preferred embodiments the assembly further comprising a water drain port fluidly communicating between the permeate lumen and the patient's urinary bladder and/or GU system.

According to still further features in the described preferred embodiments the assembly is adapted for hemodialysis.

According to still further features in the described preferred embodiments the assembly is adapted for ultrafiltration.

According to still further features in the described preferred embodiments the membrane is coated with at least one biocompatible hydrophilic material.

According to still further features in the described preferred embodiments the membrane is interleaved with at least one sheet of biocompatible hydrophilic material.

According to still further features in the described preferred embodiments the biocompatible hydrophilic material is a physiologically acceptable derivative selected from a group consisting of polyvinyl alcohol, vinyl alcohol-ethylene copolymer, polyvinyl pyrrolidone, polyhydroxyethyl acrylate, polyhydroxyethyl methacrylate, polyamide acrylate, hydroxyethyl cellulose, hydroxypropyl cellulose, chitin, chitosan, alginic acid, and gelatin or any combination thereof.

According to still further features in the described preferred embodiments the device is adapted for implantation in bone marrow having a hydrostatic pressure of about 40 mm Hg, the assembly achieving sufficient gradient pressure for filtering fluids without assistance from a pump.

According to still further features in the described preferred embodiments the device is adapted to be implanted by laporoscopic means.

According to still further features in the described preferred embodiments the device is provided with a mechanism for controlled release of a medicament into the retentate lumen.

According to still further features in the described preferred embodiments the medicament is selected from a group comprising narcotics, electrolytes and anti inflammatory agents.

According to another aspect of the present invention there is provided a method of filtering blood. The method comprises implanting a blood filtration device within a bone marrow of a subject the blood filtration device including a filter for capturing waste substances from blood flowing through the marrow and passing water and solutes to the bladder, GU system or a reservoir.

According to still further features in the described preferred embodiments the medicament the blood filtration device (BFD) comprises (i) a collector/cartridge adapted by means of size or shape to be implanted within the bone marrow of a patient; (ii) a filter material accommodated within the collector cartridge; (iii) a conduit/tube for transporting collected water to the patient's bladder or Genito-Urinary (GU) system; and, (iv) means for providing a hydrostatic pressure gradient during operation in situ between the blood flow in the bone marrow and the filter material, the pressure gradient is sufficient to direct excess water to the bladder or the GU system whilst retaining defined macromolecules;

According to still further features in the described preferred embodiments the medicament, implanting is in the bone marrow of a patient.

According to still further features in the described preferred embodiments the method further comprises applying a hydrostatic pressure gradient in situ between the blood flow in the bone marrow and the filter material sufficient to direct excess water out of the blood whilst retaining blood and defined macromolecules thereby treating end-stage renal disease.

According to still further features in the described preferred embodiments the medicament the filtering of blood is effected in a subject suffering from CHF or ESRD.

According to still further features in the described preferred embodiments the medicament implanting is effected via a minimally invasive technique.

The present invention successfully addresses the shortcomings of the presently known configurations by providing a blood filtering device capable of filtering waste products and macromolecules from blood flowing through the bone marrow.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic main illustration of the blood filtration device implanted within a patient body.

FIG. 2 is a schematic main illustration of the blood filtration device body accommodated within a cage implanted within a patient body.

FIG. 3 is a schematic illustration of an alternative view of blood filtration device for treatment of end-stage renal disease in accordance with the embodiments of the current invention.

FIG. 4 is a schematic illustration of the blood filtration device of the present invention implanted within the iliac crest bone marrow.

FIGS. 5A-C is a demonstration of images of alternative embodiments of blood filtration device.

FIG. 6 is a schematic illustration of alternative embodiments of the blood filtration device.

FIG. 7 is a schematic view illustrating the pumping motion of a bolus—like structure or mechanism which carries out self clearing of the blood filtration device.

FIG. 8 is a schematic illustration of a micro-propeller assisted mechanism for self-clearing the blood filtration device.

FIG. 9A is a schematic cross section view of the pump-assisted blood filtration device.

FIG. 9B is a schematic cross section view of the pump-assisted blood filtration device of FIG. 4A comprising a micro-propeller system.

FIGS. 10A and 10B are isometric views of a scrubber-assisted mechanism for self-clearing the blood filtration device.

FIGS. 11A and 11B are isometric views of the of a shape memory wire assisted mechanism for self-clearing the blood filtration device.

FIG. 12 is a schematic cross section view of the piezoelectrically assisted mechanism for self-clearing the blood filtration device.

FIG. 13 is a schematic illustration of a sponge-like structure which itself acts as the active filter and has a mechanism for self clearing.

FIG. 14A is a transverse section view of the implantable spirally-wound ultrafiltration membrane assembly with the external location of the hydrophilic material.

FIG. 14B is a longitudinal section view of the implantable spirally-wound ultrafiltration membrane assembly with the external location of the hydrophilic beads.

FIG. 15A is a transverse section view of the implantable spirally-wound ultrafiltration membrane assembly with the internal location of the hydrophilic beads.

FIG. 15B is a longitudinal section view of the implantable spirally-wound ultrafiltration membrane assembly with the internal location of the hydrophilic beads.

FIG. 16 is a transverse section view of the implantable elongated hollow fiber ultrafiltration membrane assembly with the external location of the hydrophilic beads.

FIG. 17 is a transverse section view of the implantable elongated hollow fiber ultrafiltration membrane assembly with the internal location of the hydrophilic beads.

FIG. 18 is a demonstration of selected embodiments of the blood filtration device implanted within a hare.

### DESCRIPTION OF THE PREFERRED EMBODIMENTS

The present invention is of a device which can be used to filter blood flowing through a bone marrow. Specifically, the present invention can be used to compensate for or correct failed kidney functions.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Numerous attempts have been made to devise dialysis devices which are free of the limitations of standard hemodialysis and peritoneal dialysis approaches.

Although some of these attempts have produced devices that can be effectively used in dialysis, non biological implants which are not limited by clotting, infection, tissue necrosis, tumor formation and thrombosis, and avoid tearing problems remain a long felt and unmet need.

The present inventors propose that a non-biological device having a filter which is implanted in the bone marrow of a subject and can be effectively used for filtering blood plasma while overcoming the above described limitations of prior art devices.

Thus, according to one aspect of the present invention there is provided a device for filtering blood plasma of a subject such as a human. Such a device can be used for blood dialysis and thus can be used to supplement or replace renal functions in ESRD and CHF.

The device (also referred to herein as a blood filtration device or BFD) includes a device body which is configured for partial or full implantation within a bone marrow of a subject and a filter disposed within or upon the device body and configured for filtering blood flowing through the bone marrow.

The device body can be attached to, or implanted within any bone via use of bone anchors, screws, staples, pins, glue and like. The device body is preferably implanted within the marrow region, it will be appreciated however, that the device body need not be fully implanted within the marrow region as long as fluid communication is established between marrow blood and the filter (i.e. that the filter is exposed to blood flowing through the bone marrow). Thus, partial implantation in which one end of device body carrying the filter resides within the marrow region and another end resides outside the bone is also envisaged by the present inventors.

Examples of bones include, but are not limited to, long bones, iliac crest, rib, sternum, hip, bones of the lower arm, and bones of the upper arm. Bones adjacent to or positioned above the patient bladder or GU system are preferred.

The device body can have any shape and any dimensions suitable for partial or full implantation within a marrow region of a bone. For example, in implantation within the iliac crest the device body can be cylindrical with a diameter of about 0.5 cm and a length of 10-20 cm or more. The device body can be made of biocompatible materials such as, for example, polyester, polypropylene, PTFE, ePTFE, PEEK, Nylon, polyether-block co-polyamide polymers, polyurethenes such as aliphatic polyether polyurethanes, PVC, PAN, PS, polyethersulfone, polyethylene, polymethylmethacrylate (PMMA), polyhydroxylmethylmethacrylate (PHMMA), thermoplastic, FEP, cellulose, extruded collagen, silicone or any combination thereof.

The present device can also include a cage structure positioned over the device body. Such a cage structure is configured for providing additional support for the device body (e.g. by further securing it to bone tissue) and/or for preventing bone ingrowth into the device body while supporting osteointegration. Such a cage can be constructed from titanium and coated with hydroxyapatite.

The filter of the present device is permeable to water and solutes and impermeable to blood cells and molecules (e.g. proteins, carbohydrates etc) above a predetermined size. The filter of the present device preferably has a cutoff size of 5-50 kDa, i.e. different configurations of the presently used filter have a pore size and arrangement which restrict molecules above 5 kDa, 10 kDa, 15 kDa, 20 kDa, 25 kDa, 30 kDa, 35 kDa, 40 kDa, 45 kDa or 50 kDa from passing through the filter. The filter is preferably configured to withstand pressures of 200 mm Hg or more without tearing and has a minimum surface area of 0.5 cm², although larger surface areas in the range of 5-10 cm² are preferred. Such surface area can be achieved by rolling the filter into rods or by folding it into a three dimensional structure.

The filter can be composed of any material suitable for such purposes, examples include metals, alloys, polymers, ceramics or combinations thereof.

Metal or alloy filters can be composed of stainless steel, nickel titanium alloys, cobalt-chrome alloys, molybdenum alloys, tungsten-rhenium alloys, or any combination thereof.

Polymeric filters can be composed of polyacrylonitrile, polysulfone, polyethersulfone, poluethylene, polymethylmethacrylate, polytetrafluoroethylene, polyester, polypropylene, polyether ether ketone, Nylon, polyether-block co-polyamide polymers, polyurethanes such as aliphatic polyether polyurethanes, polyvinyl chloride, thermoplastic, fluorinated ethylene propylene, cellulose, collagen, silicone or any combination thereof.

The filter can be of a woven or non-woven configuration. Approaches for producing woven or non-woven filters are well known in the art.

When disposed within or upon the device body, the filter can assume any configuration suitable for filtering including planar configurations, spiral configurations, pleated configurations rod configurations and the like.

The filter can be electrically and/or magnetically charged or otherwise treated in order to reduce adherence of filter-retained substances. For example, the filter can be negatively charged in order to minimize adherence of plasma proteins.

The present device further includes a fluid conduit connecting the device body to the bladder, Genito-Urinary (GU) system, or a reservoir disposed within or outside the body (e.g. collection bag). The conduit can be a tube having a diameter ranging between about 1 to about 30 mm, preferably about 5 to about 10 mm. The tube can have a length of about 5 to about 40 cm or preferably about 10 to about 20 cm.

The device body and filter are designed such that water and solutes (and molecules below the filter cutoff size) move through the filter and out of blood circulation (filter permeate) while waste substances are captured by the filter (filter retentate). Water that passes through the filter is then directed out of the body through the conduit connecting the device body to the bladder, Genito-Urinary (GU) system or reservoir. Given the fact that the flow of blood through the bone marrow ca achieve a pressure gradient of 40 mmHg or more with respect to the filter and that the filter area can be substantial especially in rod or roll configurations, a minimum of 250 cc of water per 24 hr period can be removed by the present device. Such an amount of water would be sufficient for beneficial clinical effect especially for CHF patients.

In order to enhance filtration, the present device can also include pressure increasing mechanisms such as pumps and the like, although as described herein, bone marrow blood pressure is sufficient for passive filtration without need for pressure gradient increasing mechanisms. Further description of such a mechanism is provided below with reference to the Figures.

In order to reduce filter clogging (fouling), the present device preferably includes a mechanism for clearing the surface of the filter, such a mechanism can include a pump, a scraper/scrubber, an ultrasonic emitter and the like. Further description of such a mechanism is provided below with reference to the Figures.

Matter deposited on the filter can be cleared off via such mechanism back into the bone marrow blood where it is degraded. The bone marrow is particularly suitable for such purposes since the bone marrow will filter and prevent this refuse from entering the blood stream. The material will be deposited in the bone marrow where it will undergo degradation and “clean up” by endogenous systems such as macrophages and enzymes.

The device of the present invention can also include a wireless communication unit (which can be positioned within the device body) for communicating device state and filtration rate to an extracorporeal control unit. The control unit can be used to control device operations such as filter defouling, pressure gradient (in the case of a device having an active pressure gradient mechanism such as a pump) according to data communicated from sensors positioned in or on the device body or in the conduit. Wireless communication and operation can be effected using RF, magnetic or ultrasonic communication approaches which are well known to the ordinary skilled artisan.

The device of the present invention can operate without any control over functions (dumb device), or it can operate as a closed or an open loop device. In the closed loop configuration, the implanted device can incorporate a feedback loop which adjusts the pressure gradient according to the amount of water and solutes removed from the blood. The amounts of water removed can be measured via a sensor positioned within the device body or conduit, adjustment of the pressure gradient across the filter can then be controlled via a microprocessor positioned within the device body and being in control of a pressure gradient generating mechanism such as a pump. In an open loop configuration, fluid flow sensor data can be sent to an extracorporeal processing and control unit. The processing unit can be first calibrated by a physician based on initial filtration rates. The processing unit can be recalibrated periodically (e.g. once or several times a year) if need be.

The present device may also include an indicator mechanism for alerting the subject or treating physician of filter clogging or filter failure. In the case of a device configuration including a pump mechanism, filter clogging can be detected via an increase in pump backpressure. Such an increase can be relayed wirelessly to an extracorporeal warning/control unit. Filter failure can be detected by incorporating marker dyes into the filter. Filter breakdown would result in appearance of such a dye in the urine.

Although use of filter clearing mechanism such as those described herein will ensure long duty cycle, use over extended periods of time (e.g. months, years) might necessitate filter replacement. In order to address such need, the present device preferable carries the filter in a replaceable cartridge which can be exchanged via a minimally invasive procedure. Description of such a cartridge configuration is provided hereinbelow.

The present device provides several advantages when utilized in filtration of blood plasma:

(i) bone implantation minimizes host reactions and ensures that device encapsulation is minimized;

(ii) bone implantation/anchoring maximizes device stability and minimizes movement that can lead to device failure;

(iii) positioning the filter of the device in contact with blood flowing in bone marrow negates the need for complicated blood transport conduits;

(iv) bone marrow acts as a filter to the circulatory system preventing emboli;

(v) bone marrow has an ambient pressure of 40 mm HG which can facilitate filtration (it's a naturally occurring A-V shunt);

(vi) implantation in the bone marrow is safer since it minimizes vessel to device shear and tearing; and

(vii) iliac crest positioning may allow for drainage into the bladder thus allowing the patients to urinate; and

(viii) bone marrow blood can accommodate any substances (proteins carbohydrates etc) cleared off the filter.

Specific embodiments of the present device are described in detail below with reference to the accompanying Figures.

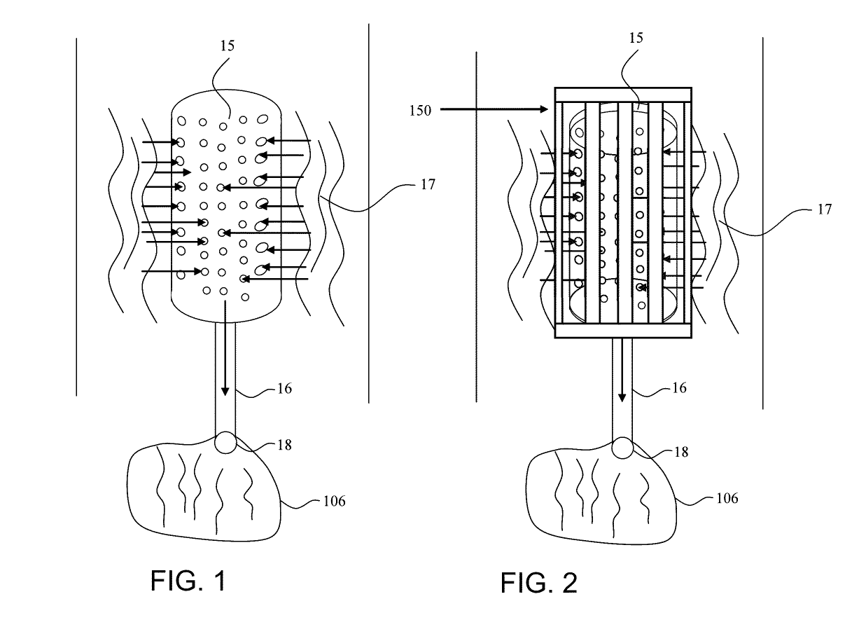

FIG. 1 schematically illustrates one embodiment of an implantable dialysis device constructed in accordance to the teachings of the present invention. In this configuration the filter 15 is positioned within cage 150.

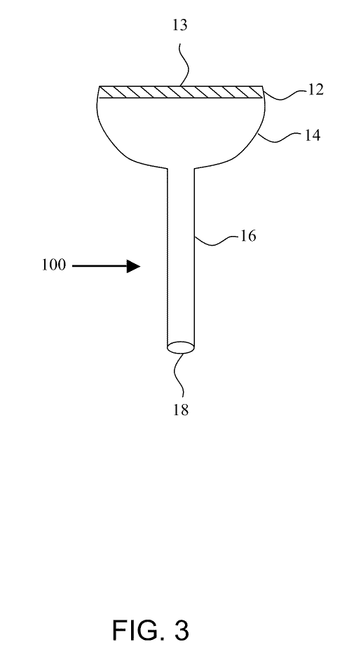

The implanted dialysis device, including cage 150 and the filter material cartridge 15, is embedded within and hence exposed to circulating blood flow 17. The aforesaid filter material cartridge 15 is substantially porous, in order to enable maximum fluid communication surface area with blood flow 17. The filtrated water flows into conduit 16 provided with a terminal 18 connected to the patient's bladder or GU system 106 so as to provide a fluid communication between the patient's marrow cavity and the bladder or GU system. In FIG. 2, cage 150 is schematically illustrated. The aforesaid cage 150 is mostly open in all directions in order to enable inflow and outflow of solutes from circulating plasma. The cage is preferably made of rigid substances such as surgical stainless steel. Thus the cage is adapted for protecting the filter material cartridge 15 from inner invasive growth of bony tissue.

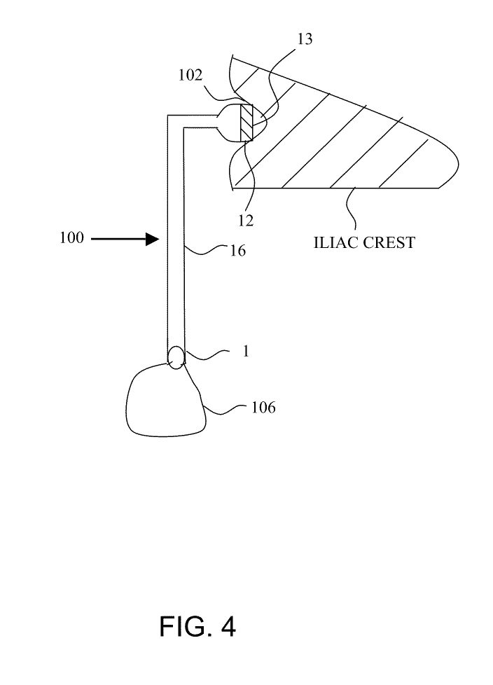

Reference is now made to FIG. 3, schematically illustrating an implantable dialysis device 100 as one of many possible alternative embodiments. Device 100 includes an implantable filter material 12 provided for selectively extracting excess water from blood under the flow of bone marrow. Implantable filter material 12 has an interface 13 which is positioned facing the marrow cavity flow when implanted. Implantable filter material 12 covers collection chamber 14 into which excess water separated from circulating plasma is collected. The collected water flows into conduit 16 then through end 18 positioned inside the patient's bladder or GU system.

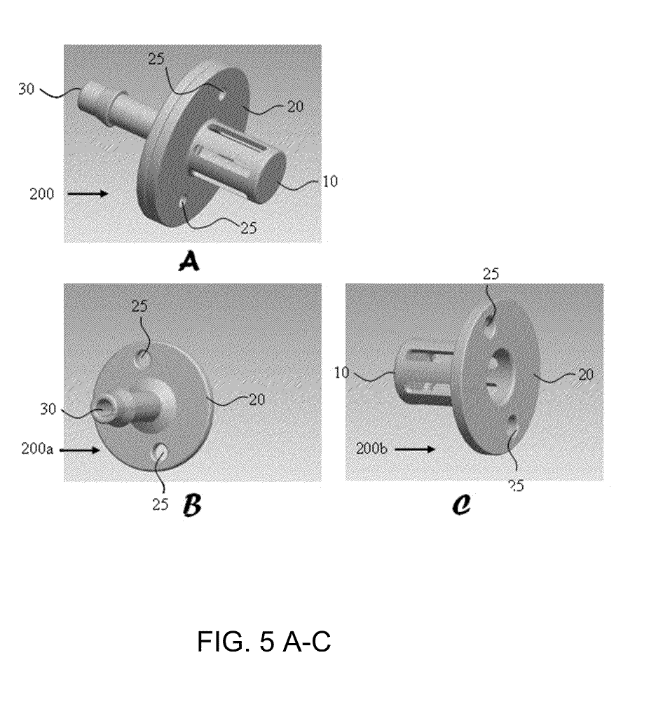

Reference is now made to FIG. 4, showing device 100 surgically implanted within the iliac crest bone marrow. Initially a cortical tissue of iliac crest region 102 is removed to thereby expose the iliac marrow cavity. Implantable filter material 12 is then connected into the iliac bone such that the filter's interface area 13 is positioned directly adjacent to or in the exposed iliac marrow cavity. Implantable filter material 12 can be affixed to the iliac bone using osteoimplantation procedures known in the art such as, but not limited to, using suitable screws, staples, anchors and sutures. Lastly, conduit end 18 is connected to bladder or GU system 106 so as to provide a fluid communication between the patient's marrow cavity and the bladder or GU system. It is a core aspect of the invention that embodiments of the device are provided suitable for implantation into the marrow of the long bones, such as the femur, tibia and sternum.

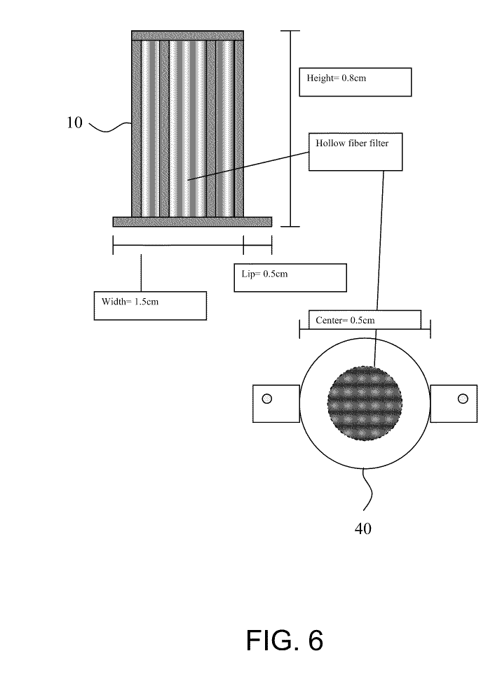

Reference is now made to FIGS. 5a-c, showing another embodiment of the present device. Device 200 includes a cage and filter material cartridge accommodated therein is implanted into the bone marrow, attached with attachment means 25. The exterior of the device comprises of titanium or plastic material and its interior is filled with polyacrimide hollow fibers. As illustrated in FIG. 5C, in one end of the device there is a housing/cage 10 accommodating filter material cartridge, facing the marrow cavity flow. The aforementioned filter material cartridge comprises polyacrimide hollow fibers used to achieve an ultra filtrate (see FIG. 5C). In the other end of the device there is an outlet drain port 30 connectable to a tube conduit/tube whose function is to collect excess fluid filtrated though the filter material structure (see FIG. 5B).

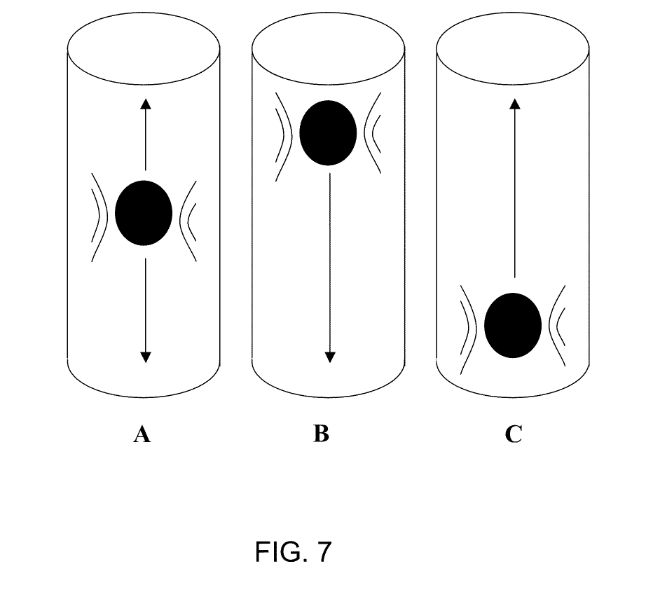

Reference is now made to FIG. 6 schematically illustrating preferred embodiments of the device. In FIG. 6 a longitudinal section view of the aforementioned cage 10 and a transverse section view of the tubing fluidly interconnected to the cage are presented. Such cage is about 0.8 cm height and about 1.5 cm width. The hollow fibers accommodated within cage 10 exit into a tube 40 which is extended through the skin and hooked up to the drain. Such tubing 40 is about 0.5 cm in diameter with an internal lumen of about 0.3 cm. All the dimensions and the relations between them which are indicated in the Figure are merely exemplary to assist in understanding aspects of an embodiment of the invention. There are embodiments of the invention whose dimensions and relations between them will be different.

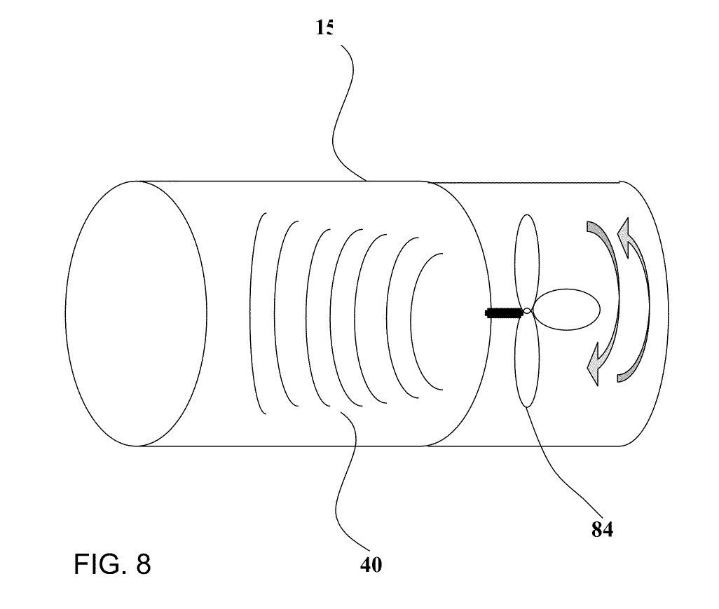

Reference is now made to FIG. 7 which schematically illustrates the pumping motion controlling the unclogging of the filter material cartridge. As can be seen a peristaltic and or pumping movement of a bolus structure within a tube, (optionally elastic) accommodating the active filter material cartridge creates a pumping motion which sucks the fluids and clots by contracting the tube, and releases them by reversibly spanning the tube.

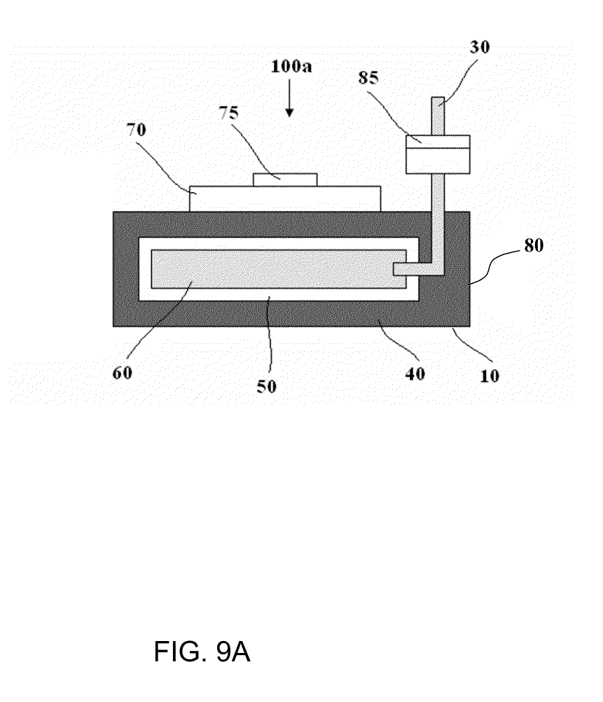

Reference is now made to FIG. 8 illustrating the self-clearing active filter assisted by a micro-propeller system. The aforesaid micro-propeller system 84 is adapted to induce alternating negative and positive pressure impulses in the permeate lumen 40 of the filter device 15. The negative and positive pressure impulses adapted to increase the hydrostatic pressure gradient both in and out of the filter and efficiently unclog the filter clots.

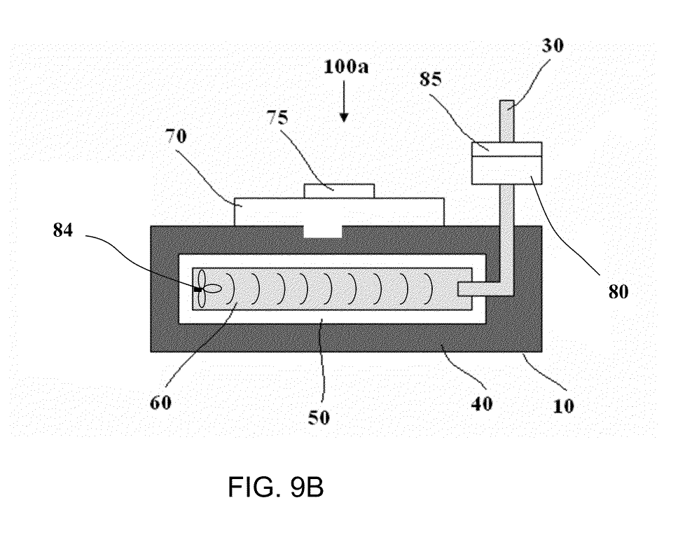

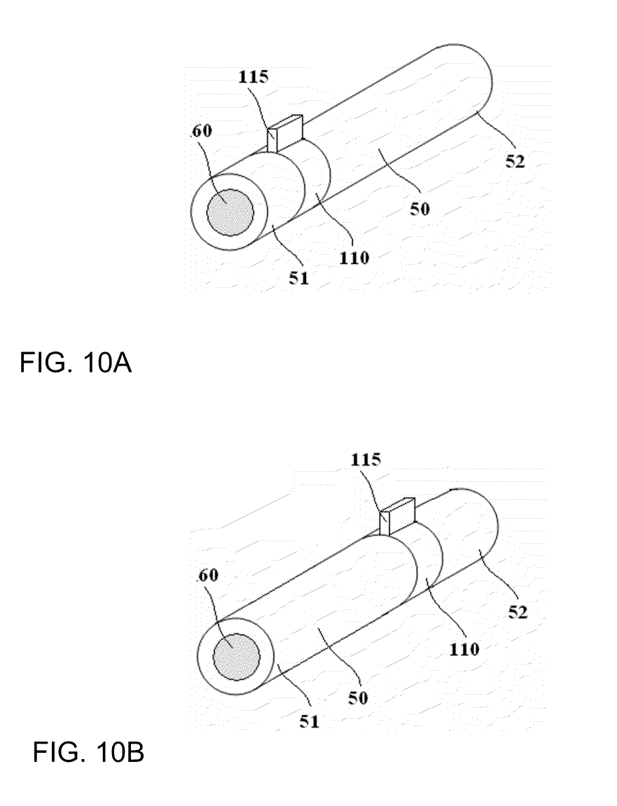

Reference is now made to FIG. 9A, presenting a pump-assisted self-clearing filter material cartridge 100a. The aforesaid filter material cartridge 100a comprises a membrane filter 50 fluidly interconnected in all directions with patient's circulating blood 40, a permeate lumen 60 fluidly connectable to the patient's urinary bladder and/or GU system through the drain port 30, Excess water contained in the blood of patient's marrow diffuses the retentate lumen 40 located outside the membrane filter structure 50 into the permeated lumen 60 inside the aforesaid structure. The filter material cartridge 100a further comprises a micro-pump 80, a micro-valve 85, a controller 70, and an electrical battery 75 enabling the active filter to be cleared. The micro-pump 80 is adapted for creating positive and/or negative hydraulic impulses in the permeate lumen 60. The micro-valve 85 is adapted for hermetically sealing the permeated lumen 60. The controller 70 activates the micro-pump 80 and micro-valve 85 according to a predetermined protocol. The electric battery 75 energizes the aforementioned controller 70, micro-pump 80, and micro-valve 85. Applying negative pressure hydraulic impulses to the membrane filter structure 50 according to the predetermined protocol decontaminates the aforesaid structure 50 and increases the filter patency.

Reference is now made to FIG. 9B, presenting the pump-assisted self-clearing filter material cartridge 100a comprising a micro-propeller system 84. The aforesaid micro-propeller system 84 is adapted for continuously inducing negative and positive pressure impulses in the permeate lumen 60, thereby increasing the hydrostatic pressure gradient and flow rate, while positive pressure can be used to clear the filter.

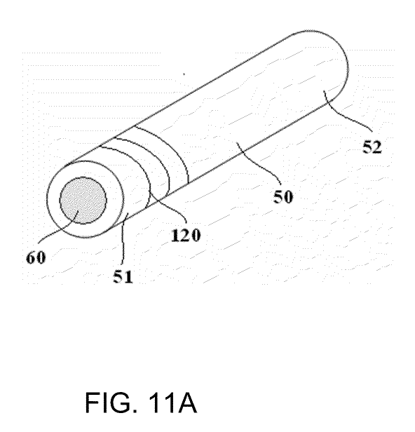

Reference is now made to FIGS. 10A and 10B, illustrating a scrubber-assisted active filter material cartridge. The aforesaid filter material cartridge comprises a ring shaped scrubber 115 and a micro-driver 110 used for removing filter clogging from the membrane filter structure 50. The aforesaid micro-driver 110 is adapted to move the ring-shaped scrubber 115 along the cylindrical membrane filter structure 50 for mechanically removing the filter clogging. The mechanical removal of the filter clogging performed according to the predetermined protocol increases the patency of the membrane filter structure 50. FIGS. 10A and 10B show displacement of the ring-shaped scrubber from one extremity 51 of the membrane filter structure 50 to another extremity thereof 52.

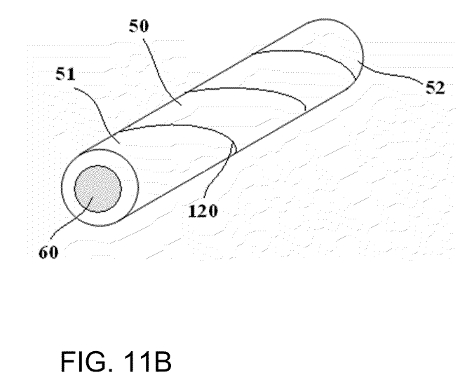

Reference is now made to FIGS. 11A and 11B, showing a scrubber based on shape memory effect. The membrane filter structure 50 is begirt by a shape memory wire 120. Applying an activating factor (for example, heating or electrical voltage) to the aforesaid wire 120 causes extending thereof along the membrane filter structure 50 and, consequently, peeling of the filter clogging at a side of the membrane filter structure 50. As seen in FIG. 6A, the wire scrubber 120 in the unextended position is localized at the one extremity of the membrane filter structure 50, for example, at the extremity 51. When activated, the wire scrubber 120 is extended to another extremity 52 of the structure 50.

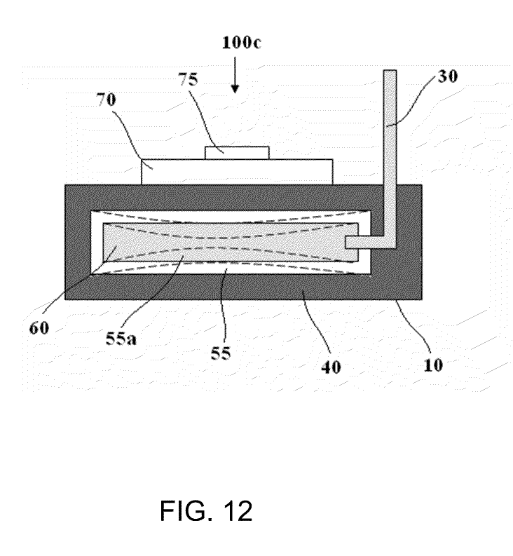

Reference is now made to FIG. 12, showing a piezoelectrically assisted active filter material cartridge 100c. The aforesaid filter material cartridge comprises a membrane filter structure 55 further comprises relaxer piezoelectric threads energizable by the controller 70 according to the predetermined protocol. The piezoelectric threads change their length in response to an applied electrical voltage.

Excess water contained in the plasma of patient's marrow diffuses from circulating blood flow 40 into the membrane filter structure 55 accommodating the permeated lumen 60 inside the aforesaid structure. Dashed lines and the numeral 55a refer to the electrically deformed membrane filter structure. It should be emphasized that the aforesaid piezoelectric threads have sufficiently high frequency response for the vibratory removal of the filter clogging of the membrane filter structure 55.

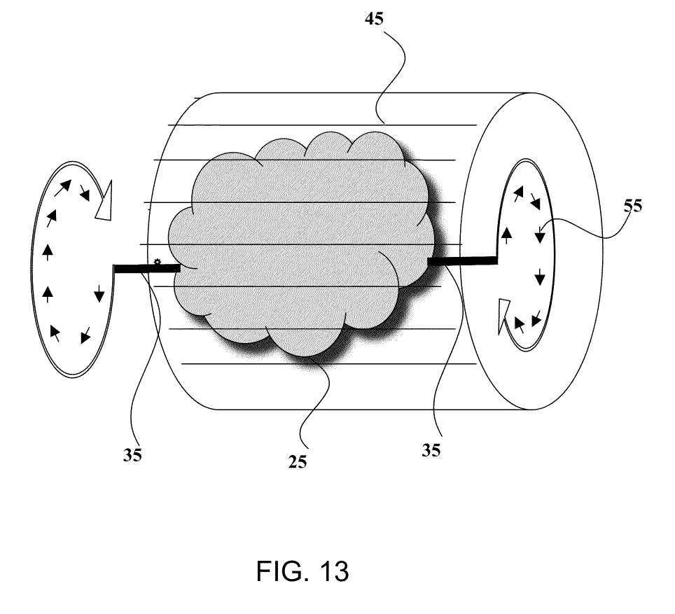

Reference is now made to FIG. 13 illustrating a sponge like structure as an embodiment of the current invention provided for creating increased hydrostatic pressure gradients and for self clearing the membrane device. The sponge-like structure is comprised of filter material 25, which is mounted by means 35 within the assembly 45. The clearing activity is assisted by the movement of means 35 in track 55, positioned at both sides of the assembly, such that a twisting or wringing or squeezing action is performed. The aforementioned action is induced or effected by powered actuating members (not shown). It will be appreciated that the preceding embodiments are exemplary and that many other embodiments of the invention are envisaged, the description herein being sufficient disclosure for a person skilled in the art to realize them.

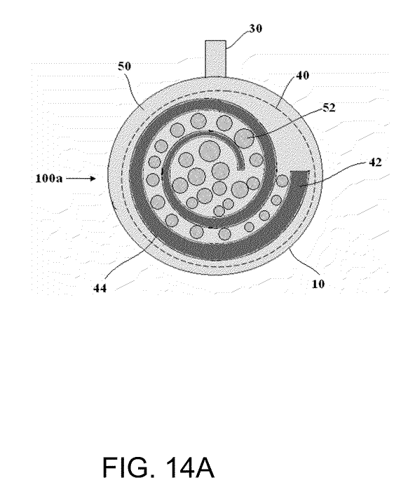

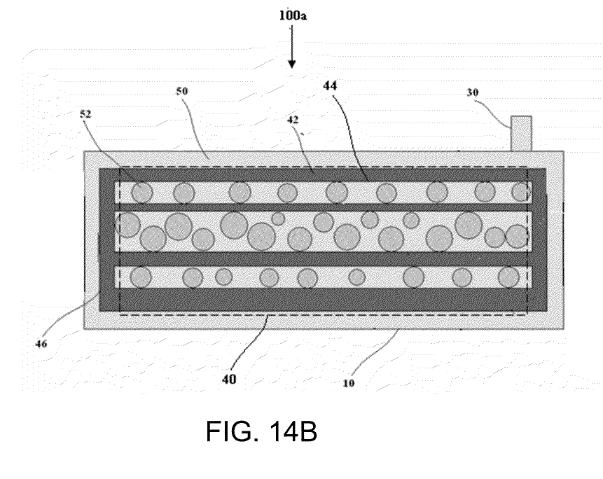

Reference is now made to FIGS. 14A and 14B, presenting transverse and longitudinal section views of the implantable filter material cartridge 100a, respectively. In accordance with one embodiment of the current invention, a spirally shaped filter cartridge 40 is disposed at the housing 10. As seen in FIG. 14A, membrane layers 44 confine a retentate lumen 42 accommodating patient's plasma flow. Excess water contained in the patient's plasma diffuses from the lumen 42 into a space 50 constituting a permeate lumen. Biocompatible hydrophilic beads 52 are partially dispersed or otherwise immobilized within the permeate lumen 50 to increase the hydrostatic pressure gradient across the membrane 42. The filtrated excess water is drained from the drain assembly through the drain port 30. The aforesaid port 30 is optionally connected to the patient's urinary bladder and/or GU system for further excess water evacuation.

Referring to FIG. 14B, the retentate lumen 42 of spirally shaped filter cartridge 45 is fluidly interconnected with circulating blood flow through the manifolds 46. The permeate lumen 50 is fluidly interconnected with the drain port 30. As said above, the excess water accommodated in the retentate lumen 42 diffuses through the filter layers into the permeate lumen 50.

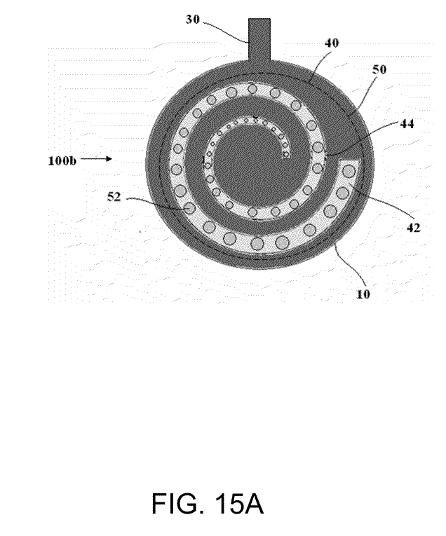

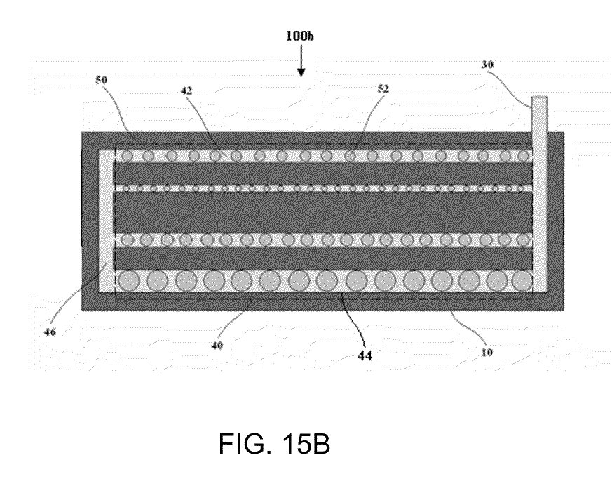

Reference is now made to FIGS. 15A and 15B, presenting a transverse section view of the implantable filter material cartridge 100b. In accordance with another embodiment of the current invention, the membrane layers 44 confine the lumen 42 accommodating the hydrophilic beads 52. Excess water contained in the patient's plasma diffuses from the retentate lumen 50 into the permeate lumen 42. Biocompatible hydrophilic beads 52 are partially dispersed or otherwise immobilized within the permeate lumen 42 to increase hydrostatic pressure gradient across the membrane 44.

Referring to FIG. 15B, the lumen 42 of the spiral structure 40 is fluidly connected to the drain port 30 while the lumen 50 is fluidly connected with circulating blood flow to reflect optimal surface area exposure. The filtrated excess water is drained from the drain assembly through the drain port 30.

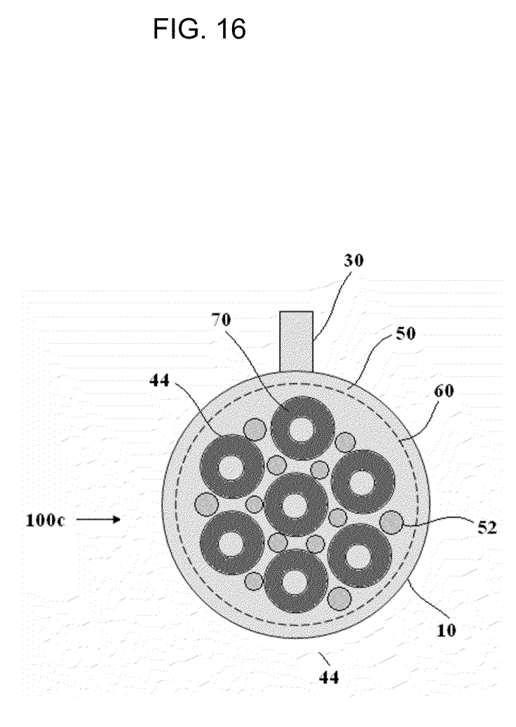

Reference is now made to FIG. 16, showing a transverse section view of the implantable filter material cartridge 100c. In accordance with a further embodiment of the current invention, a filter cartridge 60 constituting a bundle of elongated hollow membrane fibers is disposed into the housing 10. Analogously to the filter material cartridge 100a, the retentate lumen 70 of filter cartridge 60 is fluidly interconnected though out its entire surface with circulating blood flow. The permeate lumen 50 is fluidly interconnected with the drain port 30 (not shown). As in the previous examples, the excess water contained in the patient's plasma and accommodated in the retentate lumen 70 diffuses through the filer layers 42 into the permeate lumen 50. Biocompatible hydrophilic beads 52 disposed in the lumen 50 intensify the diffusion process due to increasing hydrostatic pressure gradient across the membrane 42.

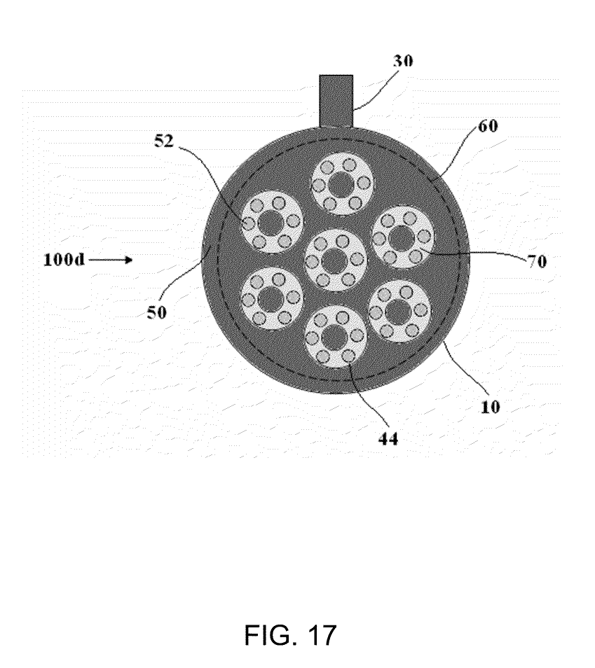

Reference is now made to FIG. 17, a transverse section view of the implantable filter material cartridge 100d. In accordance with a further embodiment of the current invention, a filter cartridge 60 constituting a bundle of elongated hollow membrane fibers is disposed into the housing 10. The lumen 70 of filter cartridge 60 is fluidly interconnected with drain port 30. The lumen 50 is fluidly interconnected though out its entire surface with circulating blood flow. As said above, the excess water contained in the patient's plasma and accommodated in the retentate lumen 50 diffuses through the filter layers 42 into the permeate lumen 70. Biocompatible hydrophilic beads 52 disposed in the lumen 70 intensify diffusion process due to increasing hydrostatic pressure gradient across the membrane 42.

As is mentioned hereinabove, the device of the present invention is highly suitable for use in treatment of fluid overload due to end stage renal disease or CHF in the patient.

Thus, the present invention also provides a method of treating end-stage renal disease or CHF. The method includes implanting within a patient in need the device disclosed herein.

It is expected that during the life of this patent many relevant filters will be developed and the scope of the term filter is intended to include all such new technologies a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above descriptions, illustrate the invention in a non limiting fashion.

### Implantation of the Present Device in Rabbits

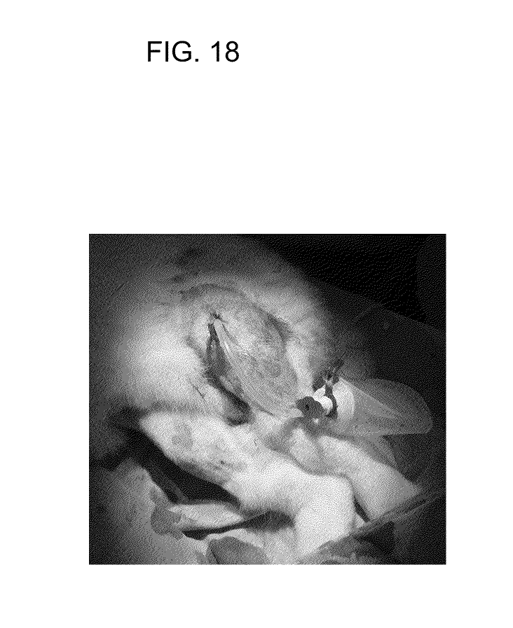

One embodiment of the present device was implanted in a bone marrow of a hare. FIG. 18 demonstrates in situ implantation of a preferred dialysis device in the bone marrow of a rabbit 300. The device used in this experiment was implanted within the bone marrow, such that an outlet port of the device body protruded out of the cortex. A silicone tube 50 was attached at one end to the outlet port, while the other end of the silicone tube was positioned extracorporeally through the skin. Tube 50 was attached to a Jackson-Pratt drain 60.

The device was implanted in 2 large healthy rabbits weighting at least 4 kilograms (female and male). The rabbits were sedated using ketamine (30 mg/kg), kasilozin (3 mg/kg) and atropine (1 mg/kg), and were anesthetized using penthotal (30 mg/kg). The skin was incised to access the femur. A drill was used to create a portal through the cortical bone and create a space in the bone marrow for filter insertion. The filter assembly (Shown in FIG. 5) was inserted with the cage end into the marrow and a silicone tube was attached to the male portion into a collecting device. The post-procedure result is shown in FIG. 18.

The rabbits were anticoagulated utilizing IV Heparin for the entire duration of the experiment. Additionally, IV fluids were administered in order to induce a temporary level of fluid overload.

In order to evaluate the ability of the device to filtrate the fluid, the rabbits were checked 4 hours post procedure. The evaluation included monitoring of the amount of fluids that are extracted through the device, signs of infection and fluid biochemistry.

The results obtained in this preliminary experiment showed that 4 hours post implantation the device extracted fluid containing creatinine, sodium and potassium from marrow blood.

This demonstrates that the implanted dialysis device of the present invention can remove excess fluid from bone marrow circulating plasma of a mammal without the need for a pump.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for filtering blood of a subject comprising:
(a) a device body configured for implantation within a bone of the subject; and
(b) a filter disposed on or within the device body and being exposed to blood flowing through a marrow of said bone, said filter being capable of filtering blood flowing through said marrow.

2. The device of claim 1, wherein said filter is impermeable to molecules above a predetermined size and permeable to water and solutes of said blood.

3. The device of claim 2, further comprising a fluid conduit connecting said device body with a bladder, a Genito-Urinary system or a reservoir.

4. The device of claim 3, wherein said conduit transports said water and solutes passed through said filter to said bladder, said Genito-Urinary or said reservoir.

5. The device of claim 1, wherein said substances are biomolecules and said filter retains biomolecules larger than 5-50 kDa.

6. The device of claim 1, further comprising a cage disposed over said device body, said cage being for protecting said device body from bone overgrowth.

7. The device of claim 6, wherein said cage is coated with titanium and/or hydroxyapatite adapted for promoting osteointegration of said device body.

8. The device of claim 1, further comprising a mechanism for increasing a blood pressure gradient across said filter.

9. The device of claim 8, wherein said mechanism is a pump.

10. The device of claim 1, further comprising a mechanism for minimizing clogging of said filter.

11. The device of claim 1, further comprising a mechanism for creating an electrical current on said device body or filter.

12. The device of claim 1, further comprising a hydrophilic material disposed within said device body, said hydrophilic material being for increasing a pressure gradient across said filter.

13. The device of claim 12, wherein said hydrophilic material is shaped as beads.

14. A method of filtering blood of a subject comprising:
(a) positioning a filter in fluid communication with blood flowing through bone marrow of the subject;
(b) retaining molecules above a predetermined size from said blood on said filter and routing a filtrate of said blood to a bladder, a Genito-Urinary system or a reservoir.

15. The method of claim 14, wherein said filtrate includes water and solutes.

16. The method of claim 14, wherein (a) is effected by implanting a device body including said filter in a bone of the subject.

17. The method of claim 14, wherein routing said filtrate of said blood to said bladder, said Genito-Urinary system or said reservoir is effected via a fluid conduit positioned between said filter and said bladder, said Genito-Urinary system or said reservoir.

18. The method of claim 17, wherein said reservoir is a bag disposed outside the body of the subject.

19. The method of claim 14, wherein said bone marrow is iliac bone marrow.

20. The method of claim 14, wherein the subject suffers from renal failure and the device is designed for enhancing or replacing kidney function.

21. The method of claim 14, wherein the subject suffers from congestive heart failure.
